(19) United States
(12) Patent Application Publication
(10) Pub. No.: US 2006/0009823 A1
(43) Pub. Date: Jan. 12, 2006

(54) Luminex® laser therapy system

(76) Inventors: Brian Dorn Richardson, Madison, CT (US); Donald Edgar Hudson, Madison, CT (US)

Correspondence Address: Brian D. Richardson, 310 Copse Rd., Madison, CT, 06443, US

(21) Appl. No.: 10/888,106

(22) Filed: Jul. 8, 2004

(51) Int. Cl. A61N 5/06

(52) U.S. Cl. 607/89

(57) ABSTRACT

Low level laser therapy apparatus for treatment of various conditions is described. In one embodiment, the apparatus includes a hand-held laser probe coupled to a control unit. The probe includes a probe head in which a laser diode(s) is mounted, emitting energy having a wavelength of 670 nm, 830 nm, 867 nm, 904 nm or 980 nm and configured to emit laser energy at a mean output power from 1 mW to 600 mW. The apparatus includes a heat-sensitive power management system allowing for continued use at high power output and audible tone to signify each joule of laser energy output. In an alternative embodiment, the apparatus includes a magnetic card reading device used in conjunction with the control unit to create single or double blinded studies with the device.

### STATEMENT REGARDING FEDERALLY SPONSORED R&D

Not Applicable

### REFERENCE TO COMPACT DISK APPENDIX

Not Applicable

### BACKGROUND OF THE INVENTION

This Invention relates generally to laser apparatus and more particularly, to low level laser therapy apparatus.

Since the 1960's, scientific studies have shown that low levels of laser energy have a non-thermal, stimulative effect on biological tissues. The therapeutic application of low level laser energy, frequently known as low level laser therapy (LLLT), or laser therapy (LT), produces beneficial clinical effects in the treatment of many musculo-skeletal, neurological and soft-tissue conditions. LT is non-invasive and has few side effects, and in the hands of a skilled practitioner can in many cases offer an alternative to surgery of drug therapies. By delivering photons of laser energy at wavelengths in the near to mid infrared range to both the surface of the skin and deep into the muscle and bone, laser therapy is used to produce a non-thermal, photochemical effect at the cellular level. The benefits of LT have enormous potential for application in human medicine, increasing the need for LT devices that can deliver the appropriate treatment to suit a particular condition.

Known LT devices and methods involve the application of laser energy at a wavelength in the near to mid-infrared range, under certain conditions which limit the dosage of laser energy being applied. Most known LT devices and methods involve the limited application of laser energy with devices having an average output below 100 mW. Such devices require extended periods of time to deliver a given dosage to a treatment point. When multiple points or large areas of the body are being treated, and when multiple treatments are needed, treatment time is a significant factor for both technician and patient.

Most known LT methods involve the application of laser energy at specific doses delivered to specific tissues, making a simple method of measuring dosage an important factor. Most known LT devices and methods also involve the application of laser energy at a limited number of available wavelengths, reducing the scope of beneficial effects that can be achieved by utilizing laser energy in both ends of the LT therapeutic window, the near to mid infrared range (about 600-1000 nm). Such LT devices limit the wavelengths and power output level available due to the rate of failure of laser diodes at the low end of the therapeutic range (600-700 nm) when operated at power levels above 100 mW for extended periods of time.

Most known LT devices also limit their suitability for research because of a lack of a simple means to blind the patient or the clinician, a necessary requirement for quality medical device research. Without the means to easily create the conditions for systematic LT research, the few studies that are completed may lack the rigorous controls needed to gain the acceptance of the wider medical community.

It would therefore be desirable to provide an LT apparatus which is capable of delivering laser light at a power output higher than 100 mW, so that treatment times are reduced and the benefits of laser energy at a higher power density can be realized. It would be further desirable to provide an LT apparatus with a means to simply keep track of the dosage of laser energy delivered, such as an audible tone that sounds each time 1 joule/cm² of laser energy is delivered to the tissue. It would be still further desirable to provide such an LT apparatus which is capable of delivering laser energy at a variety of wavelengths in the near to mid infrared range to take advantage of the specific benefits of laser energy at different wavelengths in the so-called therapeutic window, about 600-1000 nm. It would be still further desirable to provide such an LT apparatus with a heat sensitive power management system that reduces the output power from the laser probe when the probe reaches a certain temperature in order to protect the diode and reduce the risk of failures. It would be yet still further desirable to provide such an LT unit with a magnetic card reading device that allows for blinding of patients and researchers engaged in clinical studies of the benefits of laser therapy.

### BRIEF SUMMARY OF THE INVENTION

It is therefore the object of the present invention to provide an efficient and effective device for delivering the appropriate amount of laser energy at the proper wavelength to realize the potential health benefits of laser therapy.

These and other objects may be attained by a laser therapy apparatus that includes a hand-held laser probe coupled to a control unit. In one embodiment, the probe includes a probe head in which a laser diode(s) is mounted, emitting energy having a wavelength from about 670 nm to 980 nm. The laser diode used in the laser probe will depend on the wavelength desired for LT.

One unique and unobvious embodiment of the invention is selection and utilization in the hand-held laser probe of a laser diode of a wavelength of 867 nm that can reach deep into tissues. The powerful therapeutic effect is provided by the specific photodynamic properties inherent to LT.

The laser diode is mounted in the laser probe and configured to emit laser energy from 1 mW to a maximum output power of 600 mW. The laser diode is mounted in the laser probe in such a manner as to create a laser spot size of 1 cm². The laser probe includes a heat-sensitive power management system that uses a fan to constantly cool the laser probe. The heat-sensitive power management system reduces the laser output power in the event the probe reaches a certain predetermined temperature, allowing for continued use without danger of damage to the laser diode.

The control unit is an AC or battery powered box housing electronics and circuitry for controlling the operation of the LT apparatus. The control unit includes a LCD or touch screen display window for displaying the wavelength of the probe currently in use, the pulse frequency selected and, when the probe is placed in the output measurement tube, the window displays the output power of the laser probe. The display window will also display error messages to help the user identify the cause of any problem in the system's operation. The control unit also includes a button for selecting from a number of pre-set frequencies that pulse the laser energy output at a particular hertz chosen by the user for its therapeutic effect, and the associated electronics and a microprocessor storing in memory the pre-selected pulse frequency. The control unit also includes at least one power output selection mechanism such as a switch, knob or the like for pre-selecting the power level.

In one alternative embodiment, the control unit further includes a magnetic card reading device for blinding of research studies of the LT apparatus. The magnetic card reading device is affixed to the control unit with associated electronics and a microprocessor that recognizes the magnetic code on each individual card and signals the LT apparatus to deliver either true laser therapy treatment or a placebo treatment where no laser energy is emitted.

### DETAILED DESCRIPTION OF THE INVENTION

The invention is a low-level laser therapy device that includes a hand-held laser probe coupled to a control unit. The laser probe includes a probe head in which a laser diode(s) is mounted, emitting energy having a wavelength from about 670 nm to about 980 nm. The laser diode used in the laser probe depends on the desired wavelength of the laser energy emitted. Wavelengths are chosen based on factors including their depth of penetration into tissue and the type of injury and tissue being treated. Some wavelengths are more appropriate for certain conditions. For example, GaAlAs laser diodes emitting a wavelength of 670 nm, with less depth of penetration are appropriate for treating skin conditions, eye conditions and other superficial problems. However for musculoskeletal and other internal injuries, laser diodes emitting energy at about 867 nm are most appropriate due to their greater depth of penetration into tissue. In addition to wavelength, the exact number and type of laser diodes used can vary, but is limited by the requirement that the output power of the laser diode(s) be in the range of 1 mW to about 600 mW.

Therefore, in one embodiment the laser diode used is a continuously emitting GaAlAs diode emitting a wavelength in the near-infrared range of 867 nm. Higher or lower power GaAlAs diodes, or other diodes emitting in the visible to infrared range of about 670 nm to about 980 nm, including 670 nm, 830 nm, 867 nm, 904 nm and 980 nm may also be used.

In addition to wavelength, the appropriate dosage of laser energy is a crucial factor in delivering a successful laser therapy treatment. The laser probe is designed to emit an audible tone to signify each joule of laser energy emitted, with the time interval of the tone's emission changing along with adjustment of the output power level.

To reduce wear and extend the life of the laser diode, the laser probe is equipped with a heat-sensitive power management system that attenuates the power output when the probe reaches a predetermined temperature that can damage the laser diode. When the temperature of the laser probe is sufficiently reduced by the attenuation of the laser's output power the laser probe returns to normal operation.

An AC power cord with a grounded plug allows the system to be plugged into a conventional electrical outlet. In alternative embodiment the control unit is powered by a battery that can be recharged by plugging a battery charger into a conventional outlet overnight.

To control the output power of the laser energy emitted from the laser probe, the system includes a control unit with a box housing, among other things, a calibration device that communicates with the laser probe to set the output power of the laser diode between 1 and 600 mW. A cable couples the laser probe to the control unit. The control unit includes a device that displays the output power in mW, a circuit board including a control circuit, a microprocessor linked to the control circuit that stores in memory the pre-selected output power level, and a power output selection device such as a switch or knob, linked to the control circuit for pre-selecting the output power level. Generally, the control circuit functions to control the delivery of power to the laser diode according to a predetermined output power as selected using the power output selection device. In one embodiment, the power output selection device is a switch with a number of settings ranging from 1 mW to 600 mW for dialing in the selected power output. In an alternative embodiment the control unit has a liquid crystal display or a “touch screen” that allows the user to select the output power and includes a timer to set the treatment time.

In one embodiment the system is designed to recognize a variety of errors that may disrupt the normal operation of the system, such as a failure in communication between the laser probe and the control unit. Upon recognizing one of the pre-programmed errors, the system displays a message indicating the error to the user on a liquid crystal display or a touch screen on the face of the control unit.

To simplify its operation, in one embodiment the system also includes a “foot bellows” which allows for hands-free operation of the emission of laser energy from the laser probe. The foot bellows is comprised of a pressure sensitive button housed in a small pedal coupled to the control unit. When depressed, the foot bellows sends a signal to the control unit to shut off or turn on the emission of laser energy.

To allow the system to operate with a number of different laser probes that emit laser energy at wavelengths from about 670 to about 980 nm, in one embodiment the control unit includes a microprocessor that automatically detects the wavelength of the laser probe. Once it recognizes the particular wavelength in use, the system calibrates the power output measurement device to operate at the particular wavelength. All laser therapy devices are required by Federal Law to include a device for measuring the output power of the laser system when in operation. This system overcomes the difficulty that the power measurement device must be calibrated in order to operate each time a laser probe with a new wavelength is used. The system automatically detects the particular wavelength in use and sends a signal to the power measurement tube to measure the output power of the laser diode at the particular wavelength.

In one embodiment the control unit includes a data port which can be connected to a computer network in order to download treatment protocols for individual patients. After downloading the data for a particular patient the system initiates the treatment protocol, directing the user to deliver laser energy to the areas required to treat the problem. The laser system operates for the duration of the individual protocol and upon its completion shuts off the emission of laser energy and waits for the input of a new treatment protocol. This function allows the primary user of the laser system, a Medical Doctor, Chiropractor, etc., to input the treatment protocol and then leave the actual treatment to an assistant who moves the laser probe to various points on the patient according to the prompts by the system.

In one alternative embodiment of the device a magnetic card reader is used in conjunction with the control unit to allow the user to blind patients and in some cases the operator as to whether a real or a sham treatment is being delivered. The magnetic card reader is coupled to the control unit and controls whether or not laser energy is emitted from the laser probe in response to the swiping of a card with a specially coded magnetic stripe through the card reader. The cards can be given one of three separate codes, a code that initiates a “real” treatment, a code that initiates a “sham” treatment, and a code used for testing that initiates normal system operation. When a “real” card is swiped through the card reader the system operates normally and laser energy is emitted from the laser probe. When a “sham” card is swiped through the magnetic card reader the system appears to be operating normally, the emission indicator light is lit and the laser probe emits the audible tone but no laser energy is emitted. Therefore, when using a laser probe with a wavelength in the near-infrared range, not visible to the eye, it is possible to blind both the patient and the operator as to whether or nor a real laser treatment is being administered. When arranged in this way the magnetic card reader is a very useful tool in conducting double blind clinical studies, allowing for comparison of the effectiveness of real versus sham laser treatment.

In another alternative embodiment, the magnetic card reader is configured to read patient data stored on individual cards, including the condition to be treated, the patient's treatment history and the treatment protocol for that condition. When the patient is to be treated, the corresponding card is swiped through the card reader and that patient's updated information is displayed on the control unit. This allows the operator to track the patient's previous laser dosage and progress and to execute any changes in the treatment protocol. As a patient is treated over the course of a few weeks the protocols often change, reflecting differences in the condition as it responds to the treatment. When arranged in this way, the magnetic card reader and the individual patient cards allow the operator to easily keep track of the patient's progress and any change in the treatment protocol over time.

This laser therapy device is used to treat a wide range of injuries and tissues. The device delivers laser

What is claimed is:

1. Laser therapy apparatus comprising:
a probe having an aperture with a lens on one end, said probe comprising a mounted laser diode for emitting laser energy with a wavelength of 670 nm to about 980 nm, said probe configured so that the laser diode emits laser energy to a spot size of 1 cm². The laser diode contained in the laser probe will be sufficient to produce a maximum output power of 600 mW; and a control unit coupled to said probe, said control unit configured to provide power to said laser diode from either an AC power source or rechargeable battery.

2. Apparatus in accordance with claim 1 wherein said control element comprises:
an output power selection knob, switch or button for selecting the output power in the range of 1 mW to 600 mW; and
a button for selecting from a number of pre-set pulse frequencies in the range of 1 Hz to 10,000 Hz.

3. Apparatus in accordance with claim 1 wherein said laser source comprises a GaAlAs laser diode.

4. Apparatus in accordance with claim 1 wherein the wavelength of said laser energy is 867 nm.

5. Apparatus in accordance with claim 1 wherein the wavelength of said laser energy is 670 nm.

6. Apparatus in accordance with claim 1 wherein the wavelength of said laser energy is 830 nm.

7. Apparatus in accordance with claim 1 wherein the wavelength of said laser energy is 904 nm.

8. Apparatus in accordance with claim 1 wherein the wavelength of said laser energy is 980 nm.

9. Apparatus in accordance with claim 1 wherein said laser probe comprises one laser diode having a mean output power between about 1 mW and 600 mW and emitting laser energy in a beam.

10. Apparatus in accordance with claim 9 wherein said probe comprises a probe head having a forward end with an aperture, said laser diode being mounted so that the laser energy is emitted to a spot size of 1 cm².

11. Apparatus in accordance with claim 1 wherein said laser probe emits an audible tone to signify each joule of laser energy output delivered.

12. Apparatus in accordance with claim 11 wherein the time interval of said audible tone's emission changes along with any adjustment of the power level.

13. Apparatus in accordance with claim 1 wherein said laser probe further comprises a heat-sensitive power management system that constantly cools the diode during use and reduces laser output power in the event the probe reaches a certain predetermined temperature.

14. Apparatus in accordance with claim 13, wherein said heat sensitive power management system comprises a small fan mounted inside the laser probe that draws air from outside the laser probe through precision drilled holes in the body of the laser probe case to cool the laser diode.

15. Apparatus in accordance with claim 13, wherein said heat-sensitive power management system comprises associated electronics and a microprocessor in the control unit storing in memory the temperature at which it is configured to reduce power.

16. Apparatus in accordance with claim 13, wherein said heat-sensitive power management system comprises associated electronics and a microprocessor sending a signal to the laser probe to reduce power when the probe reaches the predetermined temperature.

17. Apparatus is accordance with claim 1, wherein said control unit comprises a mechanism such as a switch, knob or button which allows the user to select the output power level to settings between 1 mW and 600 mW.

18. Apparatus in accordance with claim 1, wherein said control unit comprises a button for selecting between various pre-set pulse settings between 1 Hz and 10,000 Hz.

19. Apparatus in accordance with claim 1, wherein said control unit comprises a power measurement tube used to measure the output power of the laser probe in use.

20. Apparatus in accordance with claim 19 wherein said power measurement tube comprises associated circuitry configured to automatically detect the wavelength of the laser probe in use (670 nm, 830 nm, 867 nm, 904 nm or 980 nm) and calibrate the power measurement tube to operate at that particular wavelength.

21. Apparatus in accordance with claim 1 wherein said control unit further comprises an LCD or touch screen that displays error messages related to commonly experienced errors in LT, such as bad communication between the control unit and the laser probe.

22. Apparatus in accordance with claim 21 wherein said LCD or touch screen is an input device allowing the user to select the power output from the laser probe and the treatment time.

23. Apparatus in accordance with claim 1 wherein said control unit still further comprises circuitry designed to allow connection to a computer network in order to download detailed patient and treatment information, allowing the LT treatment to be delivered by a trained assistant without direct supervision of the practitioner.

24. Apparatus in accordance with claim 23 wherein said circuitry comprises a microprocessor to store information such as patient history, condition, treatment history and treatment protocol that is communicatively coupled to a computer network through an external network port.

25. Apparatus in accordance with claim 1 wherein said control unit further comprises a foot bellows that when depressed allows for hands free operation of laser emission.

26. Apparatus in accordance with claim 25 wherein said foot bellows comprises associated electronics to communicate with the control unit and the laser probe to turn the emission of laser energy on and off.

27. An alternative embodiment of the apparatus in accordance with claim 1 wherein said control unit further comprises a magnetic card reading device to allow for the creation of blinded research with the device.

28. Apparatus in accordance with claim 27 wherein said magnetic card reading device comprises associated electronics and a microprocessor coupled to the control unit, controlling whether or not laser energy is emitted from the laser probe in response to the swiping of a card with a specially coded magnetic stripe through the card reading device.

29. Apparatus in accordance with claim 27 wherein said magnetic card reading device comprises a microprocessor that reads patient data stored on a card specifically for the individual patient and communicates that data to the control unit.
